(19) United States
(12) Patent Application Publication
(10) Pub. No.: US 2021/0198128 A1
(43) Pub. Date: Jul. 1, 2021

(54) ANTIMICROBIAL MODIFIED MATERIAL FOR TREATMENT OF FLUIDS

(71) Applicant: Research Foundation of the City University of New York, New York, NY (US)

(72) Inventors: Robert Engel, Forest Hills, NY (US); Gregory O'Mullan, Garden City, NY (US); William Blanford, Fresh Meadows, NY (US)

(21) Appl. No.: 17/055,681

(22) PCT Filed: May 15, 2019
(86) PCT No.: PCT/US19/32471

§ 371 Date: Nov. 16, 2020

Related U.S. Application Data
(60) Provisional application No. 62/671,496, filed on May 15, 2018.

(51) Int. Cl. C02F 1/50; A01N 33/12; A01N 57/20; A61L 9/00

(52) U.S. Cl. CPC C02F 1/50; A01N 33/12; C02F 2103/001; A61L 9/00; A01N 57/20

(57) ABSTRACT

A method for treating a fluid is provided. Particles are coated with quaternary ammonium or phosphonium compounds (“quats”). Fluid is passed over the particles. The biocidal properties of the quats treats the fluid.

### CROSS-REFERENCE TO RELATED APPLICATIONS

This application claims priority to and is a non-provisional of U.S. Patent Application 62/671,496 (filed May 15, 2018), the entirety of which is incorporated herein by reference.

### BACKGROUND OF THE INVENTION

There is an unmet need for efficient means of removal of microbial pollutants from fluids without addition of toxic chemicals to the subject solution or through size-exclusion filtration of that solution which is often energy intensive. There is a similar need for efficient means of avoiding microbial colonization of surfaces which may lead to degradation of those materials or compromise of fluids in contact with them or risk of exposure to infection by users of those materials or fluids.

To those ends, there are many varieties of polycationic-based antimicrobial chemicals (e.g. polyammonium such as in Engel et al. (Polycations. 2009. 18. The synthesis of polycationic lipid materials based on the diamine 1,4, diazabicyco[2.2.2]octane. Chemistry and Physics of Lipids 158(1):61-69); polyphosphonium as in Shevchenko and Engel (Shevchenko, V. and R. Engel. 1998. Polycations. III. Synthesis of polyphosphonium salts for use as antibacterial agents. Heteroatom Chemistry 9(5):495-502) and multiple means of associating these compounds with surfaces to instill antimicrobial properties into the resulting altered surface (U.S. Pat. Nos. 8,999,316; 8,470,351; 8,329,155; 7,241,453; 7,285,286). These polycationic chemicals, which are often referred to as “quats”, have been demonstrated to exhibit broad ranges of antimicrobial activity against bacteria, archaea, and protozoa as well as fungi, algae, and certain viruses (e.g. U.S. Pat. No. 8,999,316; Isquith et al. 1972. Surface-bonding antimicrobial activity of an organosilicon quaternary ammonium chloride. Applied Microbiology 24(6):859-863; and Abel et al. 2002. Preparation and investigation of antibacterial carbohydrate-based surfaces. Carbohydrate Research 337(24):2495-2499).

Current methods for treatment for the removal of viable microbes from fluids (e.g. water or air), rely primarily on six approaches: 1) size-exclusion of microbes from the advecting fluid (e.g. filter membranes, tangential filtration, hollow fiber filtration, reverse osmosis); 2) the addition, in solution, of chemical biocides or oxidants (e.g. chlorine, quats, bleach, ozone (Kahrilas G. A., J. Blotevogel, P. S. Stewart, and T. Borch. 2015. Biocides in Hydraulic Fracturing Fluids: A Critical Review of Their Usage, Mobility, Degradation, and Toxicity. Environmental Science & Technology, 49 (1)) to the subject fluid; 3) adhesion or adsorption of microbes to a stationary porous media (e.g. activated carbon (Sukdeb P., J. Joardar, and, and J. M. Song. 2006. Removal of E. coli from Water Using Surface-Modified Activated Carbon Filter Media and Its Performance over an Extended Use. Environmental Science & Technology 40 (19), 6091-6097.)); 4) electromagnetic radiation (e.g. ultraviolet (UV) Reed, 2010) of fluids; 5) contact with media columns containing toxic metals (e.g. silver or copper (Grass G., C. Rensing, and M. Solioz. 2011. Metallic Copper as an Antimicrobial Surface. Applied Environment Microbiology. March; 77(5): 1541-1547.); 6) freeze/thaw or heating of the fluid for distillation or for lysis of cells.

The discussion above is merely provided for general background information and is not intended to be used as an aid in determining the scope of the claimed subject matter.

### SUMMARY

A method for treating a fluid is provided. For example, particles are coated with quaternary ammonium or phosphonium compounds (“quats”). Fluid is passed over the particles. The biocidal properties of the quats treat the fluid.

In a first embodiment, a method for treating a fluid is provided. The method comprising: flowing a fluid through a vessel comprising a plurality of media particles, wherein each media particle in the plurality of media particles comprises a surface with an antimicrobial agent selected from a group consisting of a quaternary ammonium compound, a quaternary phosphonium compound and combinations thereof, wherein: the antimicrobial agent is silicon-free, copper-free and silver-free; each media particle in the plurality of media particles has a diameter of at least 1 mm and less than 300 mm; antimicrobial agent comprises at least one carbon chain having between 6 and 30 carbon atoms.

In a second embodiment, a method for treating water is provided. The method comprising: flowing water through a vessel, the vessel comprising a plurality of media particles, wherein each media particle in the plurality of media particles comprises a surface with a quaternary ammonium compound; wherein: the plurality of media particles are porous media particles such that the plurality of media particles produce an intrinsic permeability of 10⁻⁶ centimeters squared or greater; the quaternary ammonium compound is silicon-free, copper-free and silver-free; each media particle in the plurality of media particles has a diameter of at least 1 mm and less than 300 mm; quaternary ammonium compound comprises at least one carbon chain having between 6 and 30 carbon atoms.

In a third embodiment, a method for treating a fluid is provided. The method comprising: flowing a fluid through a vessel comprising a surface with an antimicrobial agent selected from a group consisting of a quaternary ammonium compound, a quaternary phosphonium compound and combinations thereof, wherein: the antimicrobial agent is silicon-free, copper-free and silver-free; and antimicrobial agent comprises at least one carbon chain having between 6 and 30 carbon atoms.

In a fourth embodiment, a method for storing a fluid is provided. The method comprising: storing a fluid in a vessel comprising a surface with an antimicrobial agent selected from a group consisting of a quaternary ammonium compound, a quaternary phosphonium compound and combinations thereof, wherein: the antimicrobial agent is silicon-free, copper-free and silver-free; and antimicrobial agent comprises at least one carbon chain having between 6 and 30 carbon atoms.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### DETAILED DESCRIPTION OF THE INVENTION

This disclosure pertains to the use of quaternary ammonium and phosphonium compounds (“quats”) as antimicrobial agents to treat fluids. More specifically, this disclosure pertains to antimicrobial agents that are silicon-free such that it excludes silicon-containing antimicrobial quats (e.g. 3-(trimethoxysilyl) propyldimethyloctadecyl ammonium (BIOGUARD®; SANITIZED®); Silicone Dialkyl Quats; Morais D. S., R. M. Guedes, and M. A. Lopes, 2016, Antimicrobial Approaches for Textiles: From Research to Market: Review. Materials) that are more easily hydrolyzed when associated with surfaces. This disclosure also excludes triclosan-based antimicrobials (e.g. MICROBAN®; Morais D. S., R. M. Guedes, and M. A. Lopes, 2016, Antimicrobial Approaches for Textiles: From Research to Market: Review. Materials), and silver-containing and copper-containing compounds and particles (i.e. the antimicrobial agent is triclosan-free, copper-free and silver-free).

Unlike many other antimicrobials (e.g. copper, silver, antibiotics, etc.) quats are not consumed when they interact with a microorganism or the environment. Quats do not interact with the metabolic activity of cells (e.g. such as tetracycline (Chopra I and M. Roberts. 2001. Tetracycline Antibiotics: Mode of Action, Applications, Molecular Biology, and Epidemiology of Bacterial Resistance. Microbiology and Molecular Biology Reviews. June; 65(2): 232-260.); polyhexamethylene biguanide (PHMB) (Chindera K., M. Mahato, A. K. Sharma, H. Horsley, K. Kloc-Muniak, N. F. Kamaruzzaman, S. Kumar, A. McFarlane, J. Stach, T. Bentin,8 and L. Gooda. 2016). The antimicrobial polymer PHMB enters cells and selectively condenses bacterial chromosomes. Scientific Reports. Doi: 10.1038/srep23121) nor are quats prone to promote evolution of resistant organisms (Gerba, C.P. 2015. Quaternary Ammonium Biocides: Efficacy in Application. Applied Environmental Microbiology January; 81(2): 464-469). Quats do not require cellular activity or uptake for antimicrobial action since the mechanism is impingement and physical lysis.

The method of antimicrobial action for quats is understood to occur primarily via physical disruption of the cell membrane/wall by impingement and/or electrostatic disruption causing lysis of the cell (Rutala, W. A. and D. J. Weber, 2015. Disinfection, Sterilization, and Control of Hospital Waste in Mandell, Douglas, and Bennett's Principles and Practice of Infectious Diseases (Eighth Edition)). The exact mode of action for viral species is less well constrained, but the compounds have been demonstrated to often act as virucidal against lipophilic (enveloped) viruses but are less known as a functional virucidal against hydrophilic (non-enveloped) viruses (Rutala, W. A. and D. J. Weber, 2015. Disinfection, Sterilization, and Control of Hospital Waste in Mandell, Douglas, and Bennett's Principles and Practice of Infectious Diseases (Eighth Edition), 2015; U.S. Pat. No. 8,999,316).

This technology seeks to lessen levels of viable microbes contained in gases and liquids advecting (i.e. flowing through the porous media), contained within vessels whose surfaces are coated with quats, or contained within pipe, tubes, or other agents whose surfaces are coated with quats for conveyance of fluids.

In one embodiment, a system for treating a fluid is provided. Examples of fluids include, among others, water, organic liquids, air or other gases and aqueous solutions. In another embodiment, a system for storing a fluid is provided. In one embodiment, the fluid is stored for at least 6 hours.

The media is composed of particles that may be loose or consolidated (such as in the difference between sand and sandstone). In those embodiments where the media is a porous media it has an intrinsic permeability of 10−6 centimeters (cm) squared or greater and hence does not rely on size-exclusion as the primary means of reducing microbial levels during passable air, water, or other fluids. Intrinsic permeability (Freeze, R. A., and Cherry, J. A., 1979, Groundwater: Englewood Cliffs, N.J., Prentice-Hall, 29 p.) is a property of the porous media and is independent of the properties such as dynamic viscosity and density of the fluid passing through it (Freeze, R. A., and Cherry, J. A., 1979, Groundwater: Englewood Cliffs, N.J., Prentice-Hall, 29 p.). As an example based on the empirical equation for laminar flow of an incompressible fluid passing through porous medias as formulated by Henry Philibert Gaspard Darcy in 1856 (Freeze, R. A., and Cherry, J. A., 1979, Groundwater: Englewood Cliffs, N.J., Prentice-Hall, 29 p.), the displacement pressure in centimeters of water involved in the flow of 100 cubic centimeters per second of water through the long axis of a cylinder 10 cm in diameter with a depth of 100 cm of water at sea-level and typical environmental temperatures with an intrinsic permeability of 10⁻⁶ cm² would be the displacement pressure of 1.3 meters of water which is approximately 18.5 pounds per square inch (psi).

The media may be porous or non-porous. The material being used in the subject treatment systems relies upon the lysing of microbes at the surface of the particles or walls of vessels (e.g. pipes, or other agents of fluid conveyance) as the advecting fluid encounters the surfaces of these subject items (e.g. treated gravel). It should be noted that the particles or surfaces themselves may be internally porous (e.g. gravel composed of sandstone where the sandstone has voids within), but that the internal void volume of those particles is poorly accessible (i.e. lower intrinsic permeability) and hence does not significantly contribute to microbial loss. Further, it should be noted that the total porosity of the media is a product of both the intra-particle porosity, which arises from pores within the individual particles (e.g. pores within individual sandstone gravel), and inter-particle porosity, which arises from pores between individual particles (e.g. pores between the assemblage of sandstone gravel grains). In this treatment technology, it is thought that most of the interactions between microbes and the particles or the fluid holding or conveying surfaces are at or near the surface of those items.

Examples of suitable media may include plastic media, elastomeric media, cellulosic media, epoxy media and silicate media. Examples of plastic media include, but are not limited to, three-dimensional plastic objects such as spheres, plastic membranes, polyethylene terephthalate (or polyester) (PETE or PET), high-density polyethylene (HDPE), polyvinyl chloride (PVC), low-density polyethylene (LDPE), polypropylene (PP), polystyrene (or Styrofoam) (PS), acrylonitrile butadiene, polycarbonate (PC), polylactic acid (or polylactide) (PLA), poly(methyl 2-methylpropenoate) (or acrylic) (PMMA), acetal (or polyoxymethylene, POM), styrene, fiberglass, and nylon. Examples also include polytetrafluoroethylene (PTFE) (e.g. TEFLON®), fluorinated ethylene propylene copolymers (FEP), perfluoroalkoxy (FEP, PFA), and copolymers of ethylene and tetrafluoroethylene (ETFE).

Examples of elastomeric media include natural rubbers, butyl rubber (isobutene-isoprene), chloroprene (neoprene), polychloroprene, baypren, styrene-butadieneblock copolymers, polyisoprene, polybutadiene, ethylene propylene rubber, styrene-butadiene, ethylene propylene diene rubber, silicone elastomers, halogenated butyl rubber (chlorobutyl rubber, bromobutyl rubber), fluoroelastomers (i.e. fluoropolymer elastomer), polyurethane elastomers, nitrile rubbers (including copolymer of butadiene and acrylonitrile, NBR, also called Buna N rubbers), polyurethanes, fluorosilicone, nitrilebutadiene, epichorohydrin rubber, polyacrylic rubber, silicone rubber, polyetherblock amines, chlorosulfanated polyethylene (e.g. HYPALON®), and ethylene-vinyl acetate, natural polyisoprene: cis-1,4-polyisoprene natural rubber (NR) and trans-1,4-polyisoprene gutta-percha, synthetic polyisoprene (IR for isoprene rubber), polybutadiene (BR for butadiene rubber), chloroprene rubber (CR), polychloroprene, Baypren, styrene-butadiene rubber (copolymer of styrene and butadiene, SBR, and/or copolymer of divinylbenzene and styrene), Hydrogenated Nitrile Rubbers (HNBR) such as THERBAN® and ZETPOL®, EPM (ethylene propylene rubber, a copolymer of ethylene and propylene) and EPDM rubber (ethylene propylene diene rubber, a terpolymer of ethylene, propylene and a diene-component), epichlorohydrin rubber (ECO), polyacrylic rubber (ACM, ABR), silicone rubber (SI, Q, VMQ), fluorosilicone Rubber (FVMQ), fluoroelastomers (FKM, and FEPM, TECNOFLON®, FLUOREL®, AFLAS® and DAI-EL®, perfluoroelastomers (FFKM) TECNOFLON® PFR, KALREZ®, CHEMRAZ®, PERLAST®, polyether block amides (PEBA), chlorosulfonated polyethylene (CSM), ethylene-vinyl acetate (EVA).

Examples of cellulosic media include, but are not limited to, wood, cloth, cork, chitin, cellulose derivative (cellulose esters and cellulose ethers) materials include cellulose acetate, cellulose acetate propionate, cellulose acetate butyrate, nitrocellulose, carboxymethyl cellulose, methyl cellulose, ethyl cellulose, hydroxyethyl cellulose, hydroxypropyl cellulose, hydroxypropyl methylcellulose, and LYOCELL® (a class of human-made cellulose fibers), microcrystalline cellulose, nanocelluloses, cellulose nanofibrils and cellulose nanocrystals.

Examples of silicate media include stones such as sedimentary stones and igneous stones; and glass media such as glass beads, shards, or fibers.

The system has a media (e.g. a porous or non-porous media) that comprises media particles that are 1 millimeter or greater in diameter. In various practices, the disclosed materials could have a range and variation of particle size (e.g.1 mm to 300 mm; 1 mm to 40 mm) and shapes (e.g. irregular shapes with high surface area to volume ratios). These choices impact, and hence enable, design selection of average and variation in pore sizes and shape and thus in intrinsic permeability, solute flow path length and direction, and fluid-surface interactions. In one embodiment, the media is comprised of particles with a diameter between 0.64 cm to 1.3 cm (e.g. peagravel).

In one embodiment, the quats are coated into a media that is contained within an air-stripper. Unwanted volatile chemicals or microbes are removed by pumping tainted water to the top of a large vertical vessel and “raining” it downward through the vessel. In a counter direction, air or other gas is vertically passed upward through the cascading water.

By adding quats to surfaces of porous media or the bulk material composing that media, the resulting media could then be designed for the treatment of microbial agents in fluids under a range of conditions and performance objectives. These fluids to be treated or fluid treatment components could include, but are not limited to, storm waters, industrial waters, food and beverage liquids, pharmaceutical solutions, ventilation gases, heat and volatile exchangers and numerous other systems.

The quats contain at least one carbon chain having from 6 to 30 carbon atoms. Examples of suitable quaternary ammoniums (QAs) include, but are not limited to, benzalkonium chloride (also known as alkyldimethylbenzylammonium chloride (ADBAC)), benzethonium chloride (also known as hyamine), methylbenzethonium chloride, cetalkonium chloride, cetylpyridinium chloride, cetrimonium, cetrimide, dofanium chloride, tetraethylammonium bromide, didecyldimethylammonium chloride (DDAC), dimethyldioctadecylammonium chloride and domiphen bromide. In one embodiment, the quat is a dimethylbenzylalkylammonium chloride wherein the alkyl groups are dodecyl, tetradecyl, or hexadecyl or a mixture thereof. Examples of suitable quaternary phosphoniums (QPs) include, but are not limited to, tetrakis(hexadecyl)phosphonium chloride, tetrakis(tetradecyl)phosphonium chloride, triethyl(tetradecyl)phosphonium bromide, tributylhexadecylphosphonium bromide, tributyl(dodecyl)phosphonium chloride, (1-dodecyl)triphenylphosphonium bromide, trihexyl(tetradecyl)phosphonium chloride, trihexyl(tetradecyl)phosphonium dicyanamide, tributyl(cyanomethyl)phosphonium chloride, triphenyl(tetradecyl)phosphonium bromide, hexyltriphenylphosphonium bromide, (1-octyl)triphenylphosphonium bromide, (1-tetradecyl)triphenylphosphonium bromide, (1-hexadecyl)triphenylphosphonium bromide, heptyltriphenylphosphonium bromide, dimethyl(octyl)hexadecylphosphonium chloride, and trihexyl(tetradecyl)phosphonium chloride. In one embodiment, a blend of at least two different quats is utilized.

The system works by killing microbes on contact rather than adding radiation (e.g. ultraviolet (UV)) or chemicals in solution. The porous media has an intrinsic permeability of 10⁻⁶ centimeters squared or greater and hence does not rely on size-exclusion as the primary means of reducing microbial levels during passage of or holding of air, water, or other fluids. It is readily scalable as shown in applications that range from treatment of water from a small stormwater pipe or a small building's septic system to the needs of a skyscraper or metropolitan area.

The media has surfaces that have been coated with quats which have or potentially possess or engender antimicrobial properties to the resulting item.

The surfaces have been coated with quaternary ammonium or phosphonium based organic compounds which have or potentially possess or engender antimicrobial properties to the materials for the purposes of resisting microbial colonization associated with biofouling, material degradation, oxidation, odor production, sanitation, and fomite creation.

These surfaces could include those associated with the fluid conveyance and holding of fluids such as but not limited to tubing, couplings, tanks, heat-exchange systems, baffles, valves, controls, and monitoring devices. These surfaces also include the porous media used for filtration and the surface of the vessel that store the porous consolidated or unconsolidated particles.

The antimicrobial coating used to prepare the porous media can be prepared by using a paint base (e.g. acrylic and latex). A 1:1 volume ratio of water to paint base is mixed to form a diluted paint base (e.g., in proportions such as including 250 mL base, mixed with 250 mL water). 20 mL of a 80% (w/v) solution of a quat in water is added to the diluted paint base. In another embodiment, an organic solvent-based coating (e.g. epoxy or other organic solvent as used in many waterproofing coatings) can be used as the base coating to which the solution of quat is added. In another embodiment, the coating is a plastic (e.g. low density polyethylene, polyvinyl chloride (PVC), or nylon) or an elastomeric based (e.g. polyurethane, neoprene) substance. In one embodiment, the 80% solution comprises at least two different quats. The resulting mixture agitated vigorously in a blender or by stirring. In another embodiment, the paint or coating base does not be diluted before adding quats. The mixture is then applied to the media for example by pouring over the media (e.g. gravel, plastic pellets) held in a sieve or the media can be dipped into the coating and spun to remove excess coating. The so-coated media can be spread to air dry or cured in an oven. After drying, the coated media are ready for use. As an example, the media is gravel thus coated are between 0.25 and 0.5 inches (0.64 cm to 1.3 cm) in diameter of rough shape. Rather than stones, one can use glass marbles or plastic spheres/pellets.

In one embodiment, a vessel (e.g. a column such as a tube) is packed with the media particles (such as pebbles, plastic spheres, wood, elastomers, cloth, plastic membranes, glass beads or fibers), where the surfaces have been prepared with one or more quats (e.g. a chloride anion) by coating (e.g. latex paint that has been prepared to include the quaternary ammonium salts).

In one embodiment, the media particles are placed in a vessel (e.g. a column used as antimicrobial treatment module), whereby the particles function as a treatment system for air, water, or other fluid streams and has a high intrinsic permeability, but is still able to achieve high levels of bacterial reduction. This enables treatment of a large flux of water with a minimal need for holding tanks or high-pressure pumps (that may consume excessive amounts of electrical power). A vessel of this nature allows for the high flow rate by avoiding extensive filtration techniques and does not require a holding tank for prolonged contact time. Additional modules (e.g. treatment columns), may be added to the vessel for removal of solid particles, filterables, or chemical pollutants.

The disclosed treatment system may be a component within a larger setup that may involve usage of pumps, gravity, or pressure differences to induce flow through the porous media containing antimicrobial treatment module.

The disclosed system is useful in treatment of industrial and commercial process waters, fluids, and gases. For example, the system may be used as a treatment prior to other forms of treatment such as filtration, ultrafiltration, reverse osmosis, sorption, chemical or biological treatment, or radiation.

The system can also be utilized in storm-waters, sewage-contaminated waters (e.g. receiving waterways or combined sewage and stormwater), septic discharge, removal of microbial contamination in pool and spa installations, runoff from land to water bodies, heat exchangers, heating, ventilation, and air conditioning (HVAC) filtration and evaporative coolers, for the purposes of either safe-handling, for treatment, water re-use, or for protection of the quality of receiving waters or air. These systems can be used as an initial treatment component of a larger system toward these goals or as distributed, efficacious treatment systems, as might be used for urban storm-waters or agriculture.

In heating, ventilation and air conditioning applications, evaporative coolers and heat exchangers or filters are commonly used for removal of dust, microbes, or organic chemicals and for the purposes of exchanging thermal energy or humidity (e.g. heat exchangers and evaporative coolers). Those filters are often compromised owing to the activities of various classes of microbes. The disclosed system adds quaternary ammonium or phosphonium salts that are supported on media particles, on structural or HVAC components (such as ducts, walls of venting systems, or fan blades), or into the filter itself. As an example, it would specifically include the chemical binding or coating quats to the wood fiber or other materials comprising the filters commonly used in evaporative coolers.

In thermal exchange systems water, glycol, and other liquids are commonly used. The disclosed quat treated particles and quat treated surfaces may be used for the purposes of making those surfaces and particles antimicrobial to reduce microbial levels in these fluids and to reduce the level of viable microbes in bioaerosols. Examples of such an application may include evaporative coolers where wood pads in these systems have been treated or chemically bound with quats.

After prolonged use, the media particles can be recycled. The recycling may occur by solvent washing, ion exchange, incineration, thermal regeneration, acid or chemical digestion, mechanically ablating (e.g. grinding), particle blasting (sand, walnut, etc.) and other similar processes.

The recycling process removes cellular debris following cell lysis, from microbes, organic and inorganic ions or precipitates, low-solubility organic chemicals, geologic materials or other general debris encountered during use of the media.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for treating a fluid, the method comprising:
flowing a fluid through a vessel comprising a plurality of media particles, wherein each media particle in the plurality of media particles comprises a surface with an antimicrobial agent selected from a group consisting of a quaternary ammonium compound, a quaternary phosphonium compound and combinations thereof, wherein:
the antimicrobial agent is silicon-free, copper-free and silver-free;
each media particle in the plurality of media particles has a diameter of at least 1 mm and less than 300 mm;
antimicrobial agent comprises at least one carbon chain having between 6 and 30 carbon atoms.

2. The method as recited in claim 1, wherein the media particles are plastic media, elastomeric media, cellulosic media or silicate media.

3. The method as recited in claim 1, wherein the media particles are consolidated.

4. The method as recited in claim 1, wherein the media particles are unconsolidated.

5. The method as recited in claim 1, wherein the diameter of each media particle is between 0.64 cm and 1.3 cm.

6. The method as recited in claim 1, wherein the plurality of media particles are porous media particles such that the plurality of media particles produce an intrinsic permeability of 10⁻⁶ centimeters squared or greater.

7. The method as recited in claim 1, wherein the plurality of media particles are non-porous media particles.

8. The method as recited in claim 1, wherein the antimicrobial agent is coated onto the plurality of media particles such that a surface of each media particle has the antimicrobial agent.

9. The method as recited in claim 1, wherein the fluid is an aqueous fluid.

10. The method as recited in claim 1, wherein the fluid is water.

11. The method as recited in claim 1, wherein the fluid is a gas.

12. The method as recited in claim 1, wherein the antimicrobial agent is held to the surface of each media particle by a coating.

13. The method as recited in claim 12, wherein the coating is an acrylic coating.

14. The method as recited in claim 12, wherein the coating is an organic solvent-based coating.

15. The method as recited in claim 12, wherein the coating is a plastic or epoxy coating.

16. The method as recited in claim 12, wherein the coating is an elastomer-derived coating.

17. A method for treating water, the method comprising:
flowing water through a vessel, the vessel comprising a plurality of media particles, wherein each media particle in the plurality of media particles comprises a surface with a quaternary ammonium compound; wherein:
the plurality of media particles are porous media particles such that the plurality of media particles produce an intrinsic permeability of 10⁻⁶ centimeters squared or greater;
the quaternary ammonium compound is silicon-free, copper-free and silver-free;
each media particle in the plurality of media particles has a diameter of at least 1 mm and less than 300 mm;
quaternary ammonium compound comprises at least one carbon chain having between 6 and 30 carbon atoms.

18. The method as recited in claim 17, wherein the quaternary ammonium compound is held to the surface of each media particle by an acrylic coating.

19. The method as recited in claim 17, wherein the coating is an organic solvent-based coating.

20. The method as recited in claim 17, wherein the coating is a plastic or epoxy coating.

21. The method as recited in claim 17, wherein the coating is an elastomer-derived coating.

22. A method for treating a fluid, the method comprising:
flowing a fluid through a vessel comprising a surface with an antimicrobial agent selected from a group consisting of a quaternary ammonium compound, a quaternary phosphonium compound and combinations thereof, wherein:
the antimicrobial agent is silicon-free, copper-free and silver-free; and
antimicrobial agent comprises at least one carbon chain having between 6 and 30 carbon atoms.

23. A method for storing a fluid, the method comprising:
storing a fluid in a vessel comprising a surface with an antimicrobial agent selected from a group consisting of a quaternary ammonium compound, a quaternary phosphonium compound and combinations thereof, wherein:
the antimicrobial agent is silicon-free, copper-free and silver-free; and
antimicrobial agent comprises at least one carbon chain having between 6 and 30 carbon atoms.
